(19) United States
(12) Patent Application Publication
(10) Pub. No.: US 2013/0171216 A1
(43) Pub. Date: Jul. 4, 2013

(54) AQUEOUS-BASED PERSONAL CARE PRODUCT FORMULA THAT COMBINES FRICTION-CONTROLLED FRAGRANCE ENCAPSULATION TECHNOLOGY WITH A FILM FORMING COMPOUND

(71) Applicants: Ethan Alden-Danforth, New York, NY (US); William Feuer, Hillsborough, NJ (US); Michael White, Millstone Township, NJ (US); Nancy Williams, Monroe Twp, NJ (US)

(72) Inventors: Ethan Alden-Danforth, New York, NY (US); William Feuer, Hillsborough, NJ (US); Michael White, Millstone Township, NJ (US); Nancy Williams, Monroe Twp, NJ (US)

(73) Assignee: L'Oreal USA Products, Inc., Paris (FR)

(21) Appl. No.: 13/341,048

(22) Filed: Dec. 30, 2011

(51) Int. Cl. A61K 8/11; A61Q 13/00; A61Q 5/02; A61K 8/92; A61Q 15/00

(52) U.S. Cl. 424/401; 424/65; 512/4

(57) ABSTRACT

The present disclosure relates to an aqueous microcapsule composition comprising a plurality of microcapsule particles, a film-forming mixture, and an aqueous solvent. Each microcapsule particle comprises a sol-gel material and a volatile oil-soluble active ingredient encapsulated by the sol-gel material. The film-forming mixture of the present disclosure extends the efficacy of the volatile oil-soluble active ingredient encapsulated in the microcapsule particles by improving the adherence of the microcapsule particles to the substance to which the aqueous microcapsule composition is applied (e.g., a keratinous substance or a fabric). The aqueous microcapsule composition of the instant disclosure—which is free of formaldehyde and stable in aqueous solvents—is well-suited for use in aqueous personal care products.

### TECHNICAL FIELD

The present disclosure relates to an aqueous microcapsule composition comprising a plurality of microcapsule particles and a film-forming mixture. The aqueous microcapsule composition—which is free of formaldehyde—is well suited for aqueous-based personal care products.

### BACKGROUND OF THE DISCLOSURE

Fragrance chemicals are used in numerous products to enhance the consumer's enjoyment of a product. For instance, fragrance chemicals are added to consumer products such as laundry detergents, fabric softeners, soaps, detergents, and personal care products such as, but not limited to, shampoos, body washes, deodorants, antiperspirants, and hair-styling products.

In order to enhance the effectiveness of the fragrance materials for the user, various technologies have been developed to help prevent fragrance degradation and loss. One widely used technology involves encapsulation of a fragrance material in a protective coating. The protective coating is typically a polymeric material that protects the fragrance material from evaporation, reaction, oxidation or dissipation prior to use. Examples of such polymeric encapsulated fragrance materials are disclosed in U.S. Pat. Nos. 4,081,384, 5,112,688, 5,145,842, and 6,248,703.

While encapsulation technologies have improved the tenacity of fragrance materials, most, if not all encapsulated fragrance materials either contain traces of formaldehyde and/or are starch-based. Encapsulated fragrance materials containing formaldehyde are wholly unacceptable by personal care industry standards and thus cannot be utilized in personal care products. Furthermore, starch-based encapsulations cannot be formulated into aqueous personal care products. Therefore, there is a need for a formaldehyde-free encapsulated fragrance material that can be formulated into aqueous personal care products.

### SUMMARY OF THE DISCLOSURE

The present disclosure relates to an aqueous microcapsule composition comprising a plurality of microcapsule particles, a film-forming mixture, and an aqueous solvent. Each microcapsule particle comprises a sol-gel material and a volatile oil-soluble active ingredient encapsulated by the sol-gel material. The film-forming mixture of the present disclosure extends the efficacy of the volatile oil-soluble active ingredient encapsulated in the microcapsule particles by improving the adherence of the microcapsule particles to the substance to which the aqueous microcapsule composition is applied (e.g., a keratinous substance or a fabric). The aqueous microcapsule composition of the instant disclosure—which is free of formaldehyde and stable in aqueous solvents—is well-suited for use in aqueous-based personal care products.

Accordingly, a second aspect of the disclosure relates to aqueous-based personal care products comprising the aqueous microcapsule composition described herein. In a particular aspect of the disclosure, the aqueous-based personal care product is an anti-perspirant or a deodorant comprising the aqueous microcapsule composition described herein.

Another aspect of the disclosure relates to a method of depositing the plurality of microcapsule particles on to a substrate comprising applying the aqueous microcapsule composition described herein to the substrate.

Yet another aspect of the disclosure relates to a method of scenting a substrate comprising applying an aqueous microcapsule composition of the instant disclosure to the substrate, wherein the volatile oil-soluble active ingredient of the aqueous microcapsule composition comprises a fragrance.

Another aspect of the disclosure relates to a method of treating human body odour comprising applying an aqueous microcapsule composition of the instant disclosure to a surface of human skin. In this aspect of the disclosure, the volatile oil-soluble active ingredient used in the aqueous microcapsule composition described herein comprises at least one deodorant active principle and/or at least one anti-perspirant salt or complex.

### DESCRIPTION OF BEST AND VARIOUS EMBODIMENTS OF DISCLOSURE

The term “comprising” (and its grammatical variations) as used herein is used in the inclusive sense of “having” or “including” and not in the exclusive sense of “consisting only of” The terms “a” and “the” as used herein are understood to encompass the plural as well as the singular.

The term “volatile” as used herein refers to compounds which are liquid at ambient temperature and which have a nonzero vapour pressure at ambient temperature and atmospheric pressure, ranging in particular from 0.13 Pa to 40,000 Pa (10⁻³ to 300 mmHg), in particular ranging from 1.3 Pa to 13,000 Pa (0.1 to 100 mmHg) and more particularly ranging from 1.3 Pa to 1300 Pa (0.01 to 10 mmHg).

The term “oil-soluble,” as used herein, refers to an ingredient having solubility in distilled water at ambient temperature of less than about 1 gram/100 mL. More typically, the oil-soluble ingredients mentioned herein have a solubility in distilled water at ambient temperature of less than about 0.5 grams/100 mL, and most typically have a solubility in distilled water at ambient temperature of less than about 0.1 gram/100 mL.

The term “human keratinous substance,” as used herein, is understood to mean the skin, hair, scalp, eyelashes, eyebrows, nails or lips.

The term “effective amount,” as used herein, is understood to mean the minimum quantity necessary to produce the intended or expected result.

As used herein, the expression “at least one,” means one or more and thus includes individual components as well as mixtures/combinations.

Other than in the operating examples, or where otherwise indicated, all numbers expressing quantities of ingredients and/or reaction conditions are to be understood as being modified in all instances by the term “about,” meaning within 10% to 15% of the indicated number.

A first aspect of the disclosure relates to an aqueous microcapsule composition comprising:
a. a plurality of microcapsule particles, wherein each microcapsule particle comprises:
   i. a sol-gel material; and
   ii. a volatile oil-soluble active ingredient encapsulated by the sol-gel material;
b. a film-forming mixture comprising a C₁₄₋₂₂ fatty alcohol and a C₁₂₋₂₀ alkylpolyglycoside; and
c. an aqueous solvent.

The aqueous microcapsule compositions of the instant disclosure typically comprise about 0.01 wt. % to about 6 wt. % microcapsule particles, about 0.1 wt. % to about 15 wt. % of a film-forming mixture, and about 25 wt. % to about 90 wt. % water, wherein the wt. %, in each case, is relative to the total weight of the aqueous microcapsule composition. More typically, the aqueous microcapsule compositions of the instant disclosure comprise about 0.1 wt. % to about 3 wt. % microcapsule particles, about 0.5 wt. % to about 10 wt. % of a film-forming mixture, and about 35 wt. % to about 80 wt. % water, wherein the wt. %, in each case, is relative to the total weight of the aqueous microcapsule composition. Most typically, the compositions discussed herein comprise about 0.2 wt. % to about 2 wt. % microcapsule particles, about 1 wt. % to about 5 wt. % of a film-forming mixture, and about 45 wt. % to about 70 wt. % water, wherein the wt. %, in each case, is relative to the total weight of the aqueous microcapsule composition.

The volatile oil-soluble active ingredient of the instantly disclosed aqueous microcapsule compositions is encapsulated by a sol-gel material. Sol-gel precursors (i.e., starting compounds capable of forming sol-gel material suitable for the purposes of the instant disclosure) are well known in the art. Sol-gel precursors usable in accordance with the disclosure include, for example, compounds, which are capable of forming gels, such as silicon, boron, aluminum, titanium, zinc, zirconium and vanadium. According to one aspect of the disclosure, sol-gel precursors are silicon, boron and aluminum compounds, and more particularly organo-silicon, organoboron and organoaluminum compounds. The precursors can also include metal alkoxides and b-diketonates.

Sol-gel precursors suitable for the purposes of the disclosure are selected in particular from the group of di-, tri- and/or tetrafunctional silicic acid, boric acid and alumoesters, more particularly alkoxysilanes (alkyl orthosilicates), and precursors thereof.

One example of a sol-gel precursor suitable for the purposes of the instant disclosure are alkoxysilanes corresponding to the following general formula:

(R₁O)(R₂O)M(X)(X′)

wherein X is hydrogen or —OR₃; X′ is hydrogen or —OR₄; and R₁, R₂, R₃ and R₄, independent of each other, represent a linear or branched alkyl group. More typically, R₁, R₂, R₃ and R₄, independent of each other, is a C₁₋₁₂ alkyl. M is Si, Ti or Zr.

Another example of a sol-gel precursor that is typically used in the aqueous microcapsule compositions of the instant disclosure are alkoxysilanes corresponding to the following general formula:

(R₁O)(R₂O)Si(X)(X′)

wherein X is hydrogen or —OR₃; X′ is hydrogen or —OR₄; and R₁, R₂, R₃ and R₄, independent of each other, represent a linear or branched alkyl group. More typically, R₁, R₂, R₃ and R₄, independent of each other, is a C₁₋₁₂ alkyl. M is Si, Ti or Zr.

According to one aspect of the disclosure, silicic acid esters tetramethyl orthosilicate (TMOS) and tetraethyl orthosilicate (TEOS) are used as sol-gel precursors. Typically, Dynasylan® (commercially available from Degussa Corporation, Parsippany N.J., USA) is used. Other sol-gel precursors suitable for use in the instantly disclosed aqueous microcapsule compositions are described, for instance, in German Patent Application DE 10021165. These sol-gel precursors are various hydrolysable organosilanes such as, alkylsilanes, alkoxysilanes, alkyl alkoxysilanes and organoalkoxysilanes. Besides the alkyl and alkoxy groups, other organic groups for example, allyl groups, aminoalkyl groups, hydroxyalkyl groups, etc.) may be attached as substituents to the silicon.

The particle size of each of the microcapsule particles according to the instant disclosure may be in the range of about 0.01-1000 microns in diameter. More typically, the microcapsule particles are about 0.1-100 microns in diameter, and most typically the microcapsule particles are about 1-10 microns in diameter.

The aqueous microcapsule compositions of the instant disclosure also comprise a volatile oil-soluble active ingredient. A common feature of many encapsulation processes is that they require the material to be encapsulated to be dispersed in aqueous solutions of polymers, pre-condensates, surfactants, and the like prior to formation of the microcapsule walls. Therefore, materials having low solubility in water, such as highly hydrophobic materials are typically used as the volatile oil-soluble active ingredients, as they will tend to remain in the dispersed phase and partition only slightly into the aqueous solvent of the instantly disclosued aqueous microcapsule compositions.

Examples of volatile oil-soluble active ingredients that are suitable for use in the aqueous-based compositions described herein include, but are not limited to, aromatics (e.g., fragrances), pesticides, herbicides, greasing agents, lubricants, insecticides, antimicrobial agents, pharmaceutical/therapeutic agents (e.g., an anti-infective, an antibiotic, an antibacterial agent, an anti-fungal agent, an antiviral agent, an antiparasitic agent, an anti-inflammatory agent, an anesthetic, an analgesic, an anti-allergic agent, a corticosteroid, a retinoid, an antiproliferative agent, an anticancer agent, a photodynamic therapy agent, a lubricating agent, hormones, acne active ingredients, astringent active ingredients, or a mixture thereof), nutritional ingredients (e.g., vitamins, minerals) cosmetic agents (e.g., a retinoid, an anti-wrinkle agent, a radical scavenger, a self-tanning agent, a skin whitening agent, a skin protective agent, an anti-cellulite agent, a massaging oil or an anti-wart agent, dandruff agents), deodorant active principles, anti-perspirant salt or complexes, latent heat storing agents (e.g., waxes), catalysts (e.g., organic carbonates), self-healing agents (e.g., norbornes, dicyclopentadiene), coating systems (e.g., lacquers), hydrophobic waxes, or hydrophobic solvents. Such ingredients include the ingredients described in WO 2011/110368, U.S. 2008/0050317 and U.S. 20090053301, all of which are incorporated herein by reference.

Typically, the volatile oil-soluble active ingredient is a fragrance, essential oil, plant extract, or any mixture thereof that is compatible with, and capable of being encapsulated by a monomer or a polymer.

Many types of fragrances can be utilized in the instantly disclosed aqueous microcapsule compositions so long as the fragrances are compatible with the monomer or polymer in which they are encapsulated, and the fragrances are capable of being encapsulated by the monomer or polymer. For instance, suitable fragrances include but are not limited to almond, fruits such as apple, cherry, grape, pear, pineapple, orange strawberry, raspberry; musk, flower scents such as lavender-like, rose-like, iris-like, and carnation-like. In addition, other pleasant scents such as herbal scents (e.g., rosemary, thyme, and sage), and woodland scents derived from pine, spruce and other forest smells. The instantly disclosed aqueous microcapsule compositions may also comprise fragrances derived from various oils such as essential oils, or from plant materials such as peppermint, spearmint and the like. Other familiar and popular fragrances can also be employed in the instantly disclosed compositions. Such fragrances include baby powder, popcorn, pizza, cotton candy and the like.

A list of suitable fragrances is provided in U.S. Pat. Nos. 4,534,891, 5,112,688, and 5,145,842 as well as U.S. 2010/0247660 and U.S. 20100143422—all of which are incorporated herein by reference. Another source of suitable fragrances is found in Perfumes Cosmetics and Soaps, Second Edition, edited by W. A. Poucher, 1959 (also incorporated herein by reference). Among the fragrances provided in this treatise are acacia, cassie, chypre, cylamen, fern, gardenia, hawthorn, heliotrope, honeysuckle, hyacinth, jasmine, lilac, lily, magnolia, mimosa, narcissus, freshly-cut hay, orange blossom, orchids, reseda, sweet pea, trefle, tuberose, vanilla, violet, wallflower, and the like.

The level of fragrance in the microcapsules of the instant disclosure varies from about 5 to about 95 weight percent. Typically, the level of fragrance in the microcapsule is from about 30 to about 95 weight percent, and most typically from about 50 to about 90 weight percent. The weight percent, in each case, is relative to the total dry weight of the microcapsule. Other agents can be used in conjunction with the fragrance and are understood to be included in the present disclosure.

The fragrance used in the microcapsule particles of the instant disclosure may be combined with a variety of solvents which serve to increase the compatibility of the various materials, increase the overall hydrophobicity of the blend, influence the vapor pressure of the materials, or serve to structure the blend. Solvents performing these functions are well known in the art and include mineral oils, triglyceride oils, silicone oils, fats, waxes, fatty alcohols, diisodecyl adipate, and diethyl phthalate among others.

Fragrance materials with Clog P values greater than 1, and more typically greater than 3, and most typically greater than 5 will thus result in microcapsules that will have less possibility of reacting with materials that form the capsule shell (e.g., the sol-gel materials). Surfactants contemplated for use in the present disclosure may be anionic, nonionic or cationic surfactants.

Typically, surfactants utilized in the aqueous microcapsule compositions of the instant disclosure exhibit an HLB ranging from 8 to 12. Examples of surfactants exhibiting an HLB ranging from 8 to 12 include, but are not limited to:

ethers of polyethylene glycol and/or of poly-propylene glycol and of fatty alcohols, in particular of alcohols comprising from 12 to 30 carbon atoms, typically from 14 to 22 carbon atoms, such as tridecyl alcohol, cetyl alcohol, stearyl alcohol or lauryl alcohol, the ether comprising in total from 2 to 30 oxyethylene (OE) and/or oxypropylene (OP) groups, typically from 1 to 20 oxyethylene groups and/or from 1 to 10 oxypropylene groups, such as, for example, the compounds having the INCI names steareth-8, steareth-10, steareth-16, steareth-20, ceteth-10, laureth-4, laureth-3 (such as Remcopal 121 from Ceca S.A.), trideceth-6 (such as Renex 36 from ICI Surfactants), ceteareth-5 (such as Volpo CS 5 from Croda), oleth-10 (such as Volpo N 10 from Croda), beneth-10 (such as Nikkol BB-10 from Nikkol), the ether of oleyl alcohol and of polyethylene glycol comprising 4.5 OE groups (such as, for example, Remcopal 220 from Ceca S.A.), the polyoxy-propylenated (40P)/polyoxyethylenated (1 OE) ether of cetyl alcohol (such as Nikkol PBC-31 from Nikkol) or the polyoxypropylenated (40P)/polyoxy-ethylenated (10 OE) ether of cetyl alcohol (such as Nikkol PBC-33 from Nikkol),
esters of polyethylene glycol and/or of poly-propylene glycol and of fatty acids, in particular of fatty acids comprising from 12 to 30 carbon atoms, typically from 14 to 26 carbon atoms, such as oleic acid or stearic acid, comprising from 1 to 50 oxyethylene (OE) groups, typically from 4 to 12 oxyethylene groups, such as, for example, polyethylene glycol-8 monostearate (or polyoxyethylenated 8 OE monostearate), polyethylene glycol-10 monostearate (or polyoxyethylenated 10 OE monostearate), or polyethylene glycol-12 distearate (or polyoxyethylenated 12 OE distearate),
ethers resulting from the reaction of a) polyethylene glycol and of b) esters of fatty acids (in particular of C₁₂-C₃₀ and usually C₁₂-C₂₆ acids) and of glucose which are oxyethylenated (which can comprise from 1 to 50 oxyethylene groups, usually from 4 to 10 oxyethylene groups, and from 1 to 10 glycosyl groups), such as ethers of polyethylene glycol and of esters of stearic acid and of glucose, such as polyoxyethylenated (20 OE) methyl glucose distearate,
ethers of alcohols comprising from 12 to 30 carbon atoms, typically from 14 to 22 carbon atoms, and of glycerol or of polyglycerol, the ethers comprising from 3 to 10 glycerol groups, such as, for example, polyglyceryl-3 cetyl ether, such as Chimexane NL from Chimex,
esters of fatty acids comprising from 12 to 30 carbon atoms, better still from 14 to 20 carbon atoms, such as stearic acid, and of glycerol or of polyglycerol, the esters comprising from 3 to 10 glycerol groups, such as, for example, hexa-glyceryl monostearate, it being possible for these glycerol or polyglycerol esters to comprise from 1 to 10 fatty chains;
esters of sucrose or of glucose and of fatty acids comprising from 12 to 30 carbon atoms, in particular from 14 to 20 carbon atoms; mention may be made, for example, of the mixture of esters (mono- and polyesters) of stearic acid and of sucrose sold by Croda under the reference Crodesta F110,
ethers of fatty alcohols comprising from 12 to 30 carbon atoms, in particular from 12 to 20 carbon atoms, and of sucrose or of glucose, in particular ethers of fatty alcohols comprising from 12 to 20 carbon atoms and of glucose, comprising in particular from 1.2 to 3 glucoside units, such as the compounds carrying the INCI names C12-18 alkylglucoside, C12-20 alkylglucoside (for example Montanov L from Seppic), cetearyl glucoside (such as, for example, that which is sold as a mixture with cetearyl alcohol under the reference Montanov 68 from Seppic), myristyl glucoside (such as, for example, that which is sold as a mixture with myristyl alcohol under the reference Montanov 14 from Seppic) or cetearyl glucoside (such as Tegocare CG 90 from Evonik Goldschmidt),
esters of sorbitol and/or of sorbitan and of fatty acids comprising from 12 to 30 carbon atoms, typically from 12 to 20 carbon atoms, such as lauric acid; mention may be made, for example, of sorbitan laurate, such as Span 20 from Uniqema, ethers of sorbitol and/or of sorbitan, such as ethers of beeswax and of ethoxylated sorbitan comprising from 5 to 25 OE groups, such as, for example, Sorbeth-8 beeswax or Sorbeth-20 beeswax from GBW-125 Nikko Chemical,
esters of fatty acids (in particular of C₁₂-C₃₀ and typically C₁₂-C₂₀ acids) and of oxyethylenated ethers of sorbitol and/or of sorbitan (which can comprise from 2 to 30 oxyethylene groups), such as esters of stearic acid and of sorbitol and/or of sorbitan comprising from 2 to 20 OE groups, such as polysorbate-60, polysorbate-61, sorbeth-3 isostearate, polyoxyethylenated 4 OE sorbitan monostearate or polyoxyethylenated 20 OE sorbitan tristearate,
ethers of polyethylene glycol and of cholesterol comprising from 5 to 40 oxyethylene groups, such as, for example, choleth-10 (such as Emalex CS-10 from Nihon Emulsion Company), choleth-15 (such as Emalex CS-15 from Nihon Emulsion Company) or choleth-20 (such as Emalex CS-20 from Nihon Emulsion Company),
and their mixtures.

The aqueous microcapsule compositions of the instant disclosure also comprise a film-forming mixture. The film-forming mixture comprises a C₁₄₋₂₂ fatty alcohol and a C₁₂₋₂₀ alkylpolyglycoside. For the purposes of the present disclosure, an “alkylpolyglycoside” means an alkylmonoside (degree of polymerization: 1) or alkylpolyoside (degree of polymerization: more than 1).

The alkylpolyglycosides may be used alone or in the form of mixtures of two or more alkylpolyglycosides. They conform, in general, to the following structure:

R(O)(G)ₓ

in which the radical R is a linear or branched C₁₂-C₂₂ alkyl radical, G is a saccharide residue, and x is from 1 to 5, typically from 1.05 to 2.5, and more typically from 1.1 to 2.

The saccharide residue may be selected from glucose, dextrose, sucrose, fructose, galactose, maltose, maltotriose, lactose, cellobiose, mannose, ribose, dextran, talose, allose, xylose, levoglucan, cellulose or starch. More typically the saccharide residue denotes glucose.

It should additionally be noted that each unit of the polysaccharide portion of the alkylpolyglycoside may be in L or D form, and that the configuration of the saccharide residue may be of furanoside or pyranoside type.

It is of course possible to use mixtures of alkylpolysaccharides, which may differ from one another in the nature of the alkyl moiety borne and/or in the nature of the bearing polysaccharide chain.

The alkylpolyglycoside is used in a mixture with at least one fatty alcohol, especially a fatty alcohol having from 10 to 30 carbon atoms, and more particularly from 12 to 22 carbon atoms. Examples of fatty alcohols which can be used in combination with the alkylpolyglycoside(s) of the film-forming mixtures according to the instant disclosure include linear or branched fatty alcohols of synthetic origin or else of natural origin, such as, for example, the alcohols originating from plant substances (coconut, palm kernel, palm, etc.) or animal substances (tallow, etc.). Of course, other long-chain alcohols may also be used, for example ether alcohols or else what are known as Guerbet alcohols. It is also possible to use certain longer or shorter fractions of alcohols of natural origin, for example coco (C₁₂ to C₁₆) or tallow (C₁₆ to C₁₈) or diol compounds or cholesterol.

Typically, a fatty alcohol comprising from 10 to 26 carbon atoms, and more typically from 10 to 24 carbon atoms and most typically from 12 to 22 carbon atoms is used in the film-forming mixtures of the instant disclosure.

Particular examples of fatty alcohols which can be used in the context of the present disclosure include lauryl alcohol, myristyl alcohol, cetyl alcohol, stearyl alcohol, isostearyl alcohol, palmityl alcohol, oleyl alcohol, cetearyl alcohol (a mixture of cetyl alcohol and stearyl alcohol), behenyl alcohol, erucyl alcohol, arachidyl alcohol and mixtures thereof. Most typically, cetearyl alcohol is employed. Fatty alcohols of these kinds are especially sold under the name Nafol by the company Sasol.

Moreover, it is particularly advantageous, according to the present disclosure, to employ simultaneously a fatty alcohol and an alkylpolyglycoside in which the alkyl portion is identical to that of the fatty alcohol employed. Such fatty alcohol/alkylpolyglycoside film-forming mixtures are known per se. They are described especially in patent applications WO 92/06778, WO 95/13863 and WO 98/47610 (all of which are incorporated herein by reference), and are prepared by the preparation processes indicated in these documents.

The fatty alcohol/alkylpolyglycoside mixtures typically include the products sold by SEPPIC under the Montanov names, such as the following mixtures:

cetylstearyl alcohol/cocoglucoside—Montanov 82®,
arachidyl alcohol and behenyl alcohol/arachidylglucoside—Montanov 802®,
myristyl alcohol/myristylglucoside—Montanov 14®,
cetylstearyl alcohol/cetylstearylglucoside—Montanov 68®,
C₁₄-C₂₂ alcohol/C₁₂-C₂₀ alkylglucoside—Montanov L®,
cocoalcohol/cocoglucoside—Montanov S®, and
isostearyl alcohol/isostearylglucoside—Montanov WO 18®.

The total amount of film-forming mixture in the aqueous microcapsule compositions described herein will depend on the volatile oil-soluble active ingredient used. Typically, about 0.1 wt. % to about 15 wt. % film-forming mixture is used, wherein the wt. % is relative to the total weight of the aqueous microcapsule composition. More typically, about 0.5 wt. % to about 10 wt. % is used. Most typically, about 1 wt. % to about 5 wt. % is used.

The aqueous microcapsule compositions of the instant disclosure also comprise an aqueous solvent. The aqueous solvents of the instant disclosure include water-based solvents. Most typically, the aqueous solvent is water.

The aqueous microcapsule compositions of the instant disclosure may comprise additional ingredients such as thickeners, lubricants (e.g., dimethicone), conditioners, emulsifiers, anti-perspirant active ingredients, oils, silicones, or waxes to create an emulsion, preservatives (e.g., iodopropynyl butylcarbamate), and other absorbent materials (e.g., perlite). If the compositions of the instant disclosure comprise a thickener, the thickener is typically either a hydrophilic thickener such as those described in U.S. 2011/0150805 (incorporated herein by reference) or a non-ionic thickening polymer such as those polymers mentioned in U.S. 2010/0297051 (incorporated herein by reference).

The aqueous microcapsule compositions of the instant disclosure are free of formaldehyde. Thus, the aqueous microcapsule compositions are particularly suitable for use in aqueous-based personal care products.

Accordingly, another aspect of the disclosure relates to an aqueous-based personal care product comprising the aqueous microcapsule composition of the instant disclosure. The aqueous-based personal care products of the instant disclosure may be wash-off products, which are understood to be those products that are applied for a given period of time and then are removed. These products include, but are not limited to, shampoos, conditioners, hair rinses, body washes, soaps, hand sanitizers, anti-perspirants, deodorants, perfumes, and cosmetics.

The aqueous microcapsule composition according to the disclosure can be provided in all the formulation forms conventionally used for topical application and in particular in the form of aqueous gels or of aqueous or aqueous/alcoholic solutions. They can also, by addition of a fatty or oily phase, be provided in the form of dispersions of the lotion type, of emulsions with a liquid or semiliquid consistency of the milk type, obtained by dispersion of a fatty phase in an aqueous phase (O/W) or vise versa (W/O), or of suspensions or emulsions with a soft, semisolid or solid consistency of the cream or gel type, or also of multiple (W/O/W or O/W/O) emulsions, of microemulsions, of vesicular dispersions of ionic and/or nonionic type, or of wax/aqueous phase dispersions. These aqueous microcapsule compositions are prepared according to the normal methods.

The aqueous-based personal care products of the instant disclosure include products used for the care or treatment of the skin, including the scalp, and lips, comprising an odorous substance. The aqueous microcapsule composition according to the disclosure can thus constitute a composition for scenting, caring for or treating keratinous substances and can in particular be provided in the form of eau fraiche, eau de toilette, eau de parfum, aftershave lotion, care water, silicon or aqueous/silicone care oil or anhydrous cream. It can also be provided in the form of a scented two-phase lotion (eau de toilette phase/hydrocarbon oil and/or silicon oil phase).

The disclosure also relates to aqueous microcapsule compositions packaged in a device equipped with an openwork wall, in particular a grating, packaged in a device equipped with a ball applicator (roll-on) or packaged in the form of a stick (e.g., an emulsion-based antiperspirant/deodorant stick). In this respect, the aqueous microcapsule compositions comprise the ingredients generally used in products of this type which are well known to a person skilled in the art.

Another aspect of the disclosure relates to an anti-perspirant or a deodorant comprising an aqueous microcapsule composition described herein. In a particular aspect of the present disclosure, the aqueous microcapsule compositions are incorporated into an anti-perspirant and/or deodorant roll-on aqueous-based personal care product. The anti-perspirant and/or deodorant aqueous-based personal care product contains an effective amount of the aqueous microcapsule composition of the present disclosure. In addition to comprising aqueous microcapsule compositions according to the instant disclosure, the anti-perspirant and/or deodorant aspect of the disclosure may comprise at least one deodorant active principle and/or at least one anti-perspirant salt or complex.

Within the meaning of the instant disclosure, “deodorant active principle” is understood to mean any substance capable of masking, absorbing, improving or reducing the unpleasant odour resulting from the decomposition of human sweat by bacteria. Examples of such deodorant active principles that may be used in conjunction with the instantly disclosed compositions can be found in U.S. Patent Publications 20100196484, 20050031565, 20050238598 and 20110212144, the entire disclosures of which are incorporated herein by reference.

More specifically, the deodorant active principles can be bacteriostatic agents or bactericidal agents, such as 2,4,4′-trichloro-2′-hydroxydiphenyl ether (® Triclosan), 2,4-dichloro-2′-hydroxydiphenyl ether, 3′,4′,5′-trichlorosalicylanilide, 1-(3′,4′-dichloro-phenyl)-3-(4′-chlorophenyl)urea (® Triclocarban) or 3,7,11-trimethyldodeca-2,5,10-trienol (® Farnesol); quaternary ammonium salts, such as cetyltrimethyl-ammonium salts or cetylpyridinium salts, DPTA (1,3-diaminopropanetetraacetic acid) or 1,2-decanediol (Simclariol from Symrise).

Mention may also be made, among the deodorant active principles in accordance with the disclosure, of zinc salts, such as zinc salicylate, zinc gluconate, zinc pidolate, zinc sulphate, zinc chloride, zinc lactate or zinc phenoisulphonate; chlorhexidine and its salts; sodium bicarbonate; salicylic acid and its derivatives, such as 5-(n-octanoyl)salicylic acid; glycerol derivatives, such as, for example, caprylic/capric glycerides (Capmul MCM from Abitec), glycerol caprylate or caprate (Dermosoft GMCY and Dermosoft GMC respectively from Straetmans) or polyglyceryl-2 caprate (Dermosoft DGMC from Straetmans); biguanide derivatives, such as polyhexamethylene-biguanide salts; silver, zeolites or silver-free zeolites.

In the event of incompatibility or in order to stabilize them, some of the active principles mentioned above can be incorporated in spherules, in particular vesicles, which may be ionic or nonionic, and/or nanoparticles (nanocapsules and/or nanospheres).

The deodorant active principles are usually present in the aqueous microcapsule compositions according to the disclosure in concentrations ranging from 0.01 to 5% by weight, relative to the total weight of the composition.

In order to improve the antiperspirant effectiveness of the composition, use may additionally be made of one or more water-soluble anionic polymers comprising a Bronsted acid, in particular those deriving from maleic acid and/or maleic anhydride which are described in Patent Application WO 02/49590.

Furthermore, “anti-perspirant salt or complex,” as used in the instant disclosure refers to any salt or complex which, by itself alone, has the effect of reducing or limiting the flow of sweat and/or absorbing human sweat. Examples of such anti-perspirant salt or complexes can be found in the OTC final monograph on Antiperspirant Actives and U.S. Patent Publications 20100196484, 20050031565, 20050238598, and 20110212144, the entire disclosures of which are incorporated herein by reference.

The antiperspirant salts or complexes in accordance with the disclosure are generally chosen from aluminium and/or zirconium salts or complexes. They are typically chosen from aluminium hydrohalides; aluminium zirconium hydrohalides, or complexes of zirconium hydroxychloride and of aluminium hydroxychloride, with or without an amino acid, such as those described in U.S. Pat. No. 3,792,068.

Mention may in particular be made, among the aluminium salts, of aluminium chlorohydrate in the activated or nonactivated form, aluminium chlorohydrex, the aluminium chlorohydrex polyethylene glycol complex, the aluminium chlorohydrex propylene glycol complex, aluminium dichlorohydrate, the aluminium dichlorohydrex polyethylene glycol complex, the aluminium dichlorohydrex propylene glycol complex, aluminium sesquichlorohydrate, the aluminium sesquichlorohydrex polyethylene glycol complex, the aluminium sesquichlorohydrex propylene glycol complex or aluminium sulphate buffered with sodium aluminium lactate.

Mention may in particular be made, among aluminium zirconium salts, of aluminium zirconium octachloro-hydrate, aluminium zirconium pentachlorohydrate, aluminium zirconium tetrachlorohydrate or aluminium zirconium trichlorohydrate.

The complexes of zirconium hydroxychloride and of aluminium hydroxychloride with an amino acid are generally known under the name ZAG (when the amino acid is glycine). Mention may be made, among these products, of the aluminium zirconium octachlorohydrex glycine, aluminium zirconium pentachlorohydrex glycine, aluminium zirconium tetrathiorohydrex glycine and aluminium zirconium trichlorohydrex glycine complexes.

Use will more particularly be made of aluminium chlorohydrate in the activated or nonactivated form.

The antiperspirant salts or complexes can be present in the aqueous microcapsule composition according to the disclosure in a concentration of approximately 0.5 to 25% by weight, relative to the total weight of the aqueous microcapsule composition.

Another aspect of the disclosure relates to a method of depositing the plurality of microcapsule particles of the aqueous microcapsule composition described herein on a substrate. The term “substrate,” as used herein, includes, but is not limited to, human keratinous substances, fabric or textile materials (e.g., garments, clothes, towels, sheets, accessories, hats, gloves, outerwear, curtains, rugs, or stuffed animals), or hard surfaces (e.g., wood, tile, linoleum, countertops, or dishes). The microcapsule particles are deposited by applying the aqueous microcapsule composition of the instant disclosure to the desired substrate. A particular aspect of the present disclosure involves applying the aqueous microcapsule composition described herein on a substrate during washing and/or rinsing processes (e.g., during a washing or rinsing cycle for a clothes or dishwasher).

Once deposited, the microcapsules release the volatile oil-soluble active ingredient contained therein through small cracks or imperfections in the microcapsule wall caused by drying, physical or mechanical means, or by large-scale rupture of the microcapsule wall. In each of these cases, the volatility of the encapsulated volatile oil-soluble active ingredient is critical to both the speed and duration of release, which in turn control consumer perception. Thus, volatile oil-soluble active ingredients which have higher volatility as evidenced by normal boiling points of less than 250° Celsius, and more typically less than about 225° Celsius are used in cases where quick release and impact of the volatile oil-soluble active ingredient is desired. In contrast, volatile oil-soluble active ingredients that have lower volatility (e.g., having boiling points greater than 225° Celsius) are used when a longer duration of the volatile oil-soluble active ingredient is desired. Volatile oil-soluble active ingredients of the instant disclosure may be combined in any proportion to achieve the desired speed and duration of perception.

A particular aspect of the present disclosure relates to a method of scenting or fragrancing a substrate comprising applying an aqueous microcapsule composition described herein to the substrate. In order to provide the highest fragrance impact from the microcapsule particles deposited on the various substrates referenced above, materials with a high odor-activity are typically used as the volatile oil-soluble active ingredient. Materials with high odor-activity can be detected by sensory receptors at low concentrations in air, thus providing high fragrance perception from low levels of deposited capsules. This property must be balanced with the volatility as described above. Some of the principles mentioned above are disclosed in U.S. Pat. No. 5,112,688. Examples of fragrance ingredients that may be used as volatile oil-soluble active ingredients in the microcapsule particles of the instant disclosure include, but are not limited to, the fragrance ingredients listed in Table 1 of U.S. 2010/0143422 which is incorporated herein by reference.

Usually, higher ClogP materials are used, meaning that those materials with a ClogP value of 4.5 are employed more typically than those fragrance materials with a ClogP of 4, and those materials are used more typically than fragrance materials with a ClogP of 3.3.

When used to scent or fragrance a substrate, the volatile oil-soluble active ingredient of the aqueous microcapsule compositions described herein should comprise at least about 40 weight percent of materials with ClogP greater than 3.3, more typically greater than about 80 weight percent of materials and most typically greater than about 90 weight percent of materials with ClogP greater than 4 wherein the weight percent, in each case, is based on the total dry weight of the microcapsule particle. In an additional embodiment, the aqueous microcapsule composition may contain a volatile oil-soluble active ingredient comprising fragrance materials with a ClogP greater than about 1.5.

Those with skill in the art appreciate that fragrance formulations are frequently complex mixtures of many fragrance ingredients. A perfumer commonly has several thousand fragrance chemicals to work from. Those with skill in the art appreciate that the aqueous microcapsule compositions of the instant disclosure may contain a single ingredient, but it is much more likely that the compositions described herein will comprise at least eight or more fragrance chemicals, more likely to contain twelve or more and often twenty or more fragrance chemicals. The present disclosure also contemplates the use of complex fragrance formulations containing fifty or more fragrance chemicals, seventy five or more or even a hundred or more fragrance chemicals in a fragrance formulation.

Usually, fragrance materials used as the volatile oil-soluble active ingredient will have both high ClogP and high vapor pressure. Among those having these properties are: Para cymene, Caphene, Mandarinal Firm, Vivaldie, Terpinene, Verdox, Fenchyl acetate, Cyclohexyl isovalerate, Manzanate, Myrcene, Herbavert, Isobutyl isobutyrate, Tetrahydrocitral, Ocimene and Caryophyllene.

Yet another aspect of the disclosure relates to a method of treating (e.g., a method of inhibiting, preventing, or reducing) human body odours comprising applying an effective amount of an aqueous microcapsule composition described herein to a surface of human skin. The human skin may be, for example, armpit skin or foot skin. When used to treat human body odours, the volatile oil-soluble active ingredient comprises at least one deodorant active principle and/or at least one anti-perspirant salt or complex.

The following examples are provided as specific aspects of the present disclosure. These and additional modifications and improvements of the present disclosure may also be apparent to those with ordinary skill in the art. The particular combinations of elements described and illustrated herein are intended only to represent certain aspects of the present disclosure and are not intended to serve as limitations of alternative articles within the spirit and scope of the disclosure.

### Example 1

### Antiperspirant/Deodorant Aqueous Microcapsule Composition for Roll-on

An antiperspirant/deodorant aqueous microcapsule composition to be incorporated in a ball-top dispenser of the roll-on type was prepared by adding by small portions a mixture comprising:

274.13 grams of Water

5.00 grams Steareth-100/PEG-136/HDI Copolymer

150.00 grams Aluminum Chlorohydrate

35.00 grams Dimethicone

15.00 grams C14-22 Alcohols and C12-20 Alkyl Glucoside

0.38 grams Iodopropynyl Butylcarbamate

5.00 grams Perlite

7.50 grams Fragrance

8.00 grams Encapsulated Fragrance

The mixture was formed from two phases as described below.

PHASE A: Steareth-100/PEG-136/HDI copolymer was sprinkled on hot water (i.e., water having a temperature of approximately 80° Celsius) contained in a vessel for 30 minutes under homogenizing agitation. Aluminum chlorohydrate solution was then added to the vessel containing the steareth-100/PEG-136/HDI copolymer/water mixture. The vessel was heated, using a hot water bath, to approximately 80° Celsius, and the mixture was stirred until uniform.

PHASE B: Dimethicone and the C14-22 Alcohols and C12-20 Alkyl Glucoside mixture were mixed in a vessel, and the vessel was heated, using a hot water bath, to approximately 80° Celsius.

The contents of the vessel used in PHASE B were then introduced to the vessel containing the PHASE A contents with UltraTurrax stirring. The contents of the PHASE B vessel were mixed with the contents of the PHASE A vessel for approximately 20 minutes. After 20 minutes elapsed, the vessel containing the PHASE A and PHASE B contents was removed from the hot water bath and the contents of the vessel were mixed using sweep blade mixing. Once the vessel reached approximately 55° Celsius, iodopropynyl butylcarbamate and perlite were added. The contents of the vessel were then mixed until uniform. The vessel was then cooled to a temperature of approximately 25° Celsius, and free fragrance and encapsulated fragrance were added. The contents of the vessel were then mixed until a uniform mixture was formed.

All publications cited in the specification, both patent publications and non-patent publications are indicative of the level of skill of those skilled in the art to which this disclosure pertains. Any publication not already incorporated by reference herein is herein incorporated by reference to the same extent as if each individual publication were specifically and individually indicated as being incorporated by reference.

Still other objects and advantages of the present disclosure will become readily apparent by those skilled in the art from the preceding detailed description, wherein it is shown and described preferred embodiments, simply by way of illustration of the best mode contemplated. As will be realized the disclosure is capable of other and different embodiments, and its several details are capable of modifications in various obvious respects, without departing from the disclosure. Accordingly, the description is to be regarded as illustrative in nature and not as restrictive.

What is claimed is:

1. An aqueous microcapsule composition comprising:
a. a plurality of microcapsule particles, wherein each microcapsule particle comprises:
i. a sol-gel material; and
ii. a volatile oil-soluble active ingredient encapsulated by the sol-gel material;
b. a film-forming mixture comprising a C₁₄₋₂₂ fatty alcohol and an C₁₂₋₂₀ alkylpolyglycoside; and
c. an aqueous solvent.

2. The aqueous microcapsule composition according to claim 1, wherein the composition comprises:
a. about 0.01 wt. % to about 6 wt. % of the microcapsule particles;
b. about 0.1 wt. % to about 15 wt. % of the film-forming mixture; and
c. about 25 wt. % to about 90 wt. % water,
wherein the wt. %, in each case, is relative to the total weight of the aqueous microcapsule composition.

3. The aqueous microcapsule composition according to claim 1, wherein the film-forming mixture is a mixture selected from the group consisting of a cetylstearyl alcohol/cocoglucoside mixture, an arachidyl alcohol and behenyl alcohol/arachidylglucoside mixture, a myristyl alcohol/myristylglucoside mixture, a cetylstearyl alcohol/cetylstearylglucoside mixture, a C₁₄-C₂₂ alcohol/C₁₂-C₂₀ alkylglucoside mixture, a cocoalcohol/cocoglucoside mixture, and an isostearyl alcohol/isostearylglucoside mixture.

4. The aqueous microcapsule composition according to claim 1, wherein the sol-gel material is a metal or semi-metal alkoxide monomer, a metal ester monomer, a semi-metal ester monomer or an alkoxysilanes monomer corresponding to the formula:
(R₁O)(R₂O)M(X)(X′),
wherein M is Si, Ti, or Zr; X is hydrogen or —OR₃; X′ is hydrogen, or —OR₄; and R₁, R₂, R₃ and R₄, independent of each other, represent a linear or branched alkyl group.

5. The aqueous microcapsule composition according to claim 1, wherein the sol-gel material is tetramethyl orthosilicate, tetraethyl orthosilicate, or a mixture thereof.

6. The aqueous microcapsule composition according to claim 1, wherein the aqueous solvent is water.

7. The aqueous microcapsule composition according to claim 1, wherein the film-forming mixture is a C₁₄-C₂₂ alcohol/C₁₂-C₂₀ alkylglucoside mixture and the aqueous solvent is water.

8. The aqueous microcapsule composition according to claim 1, wherein the composition is free of formaldehyde.

9. The aqueous microcapsule composition according to claim 1, wherein the volatile oil-soluble active ingredient comprises a fragrance.

10. A method of scenting a human keratinous substance comprising applying the aqueous microcapsule composition according to claim 9 to the human keratinous substance.

11. The aqueous microcapsule composition according to claim 9, wherein the volatile oil-soluble active ingredient further comprises at least one deodorant active principle and/or at least one antiperspirant active principle.

12. A method of treating a human body odour comprising applying an effective amount of the aqueous microcapsule composition according to claim 11 to a surface of human skin.

13. The method according to claim 12, wherein the effective amount of the aqueous microcapsule composition is applied to a surface of human armpit skin.

14. The method according to claim 12, wherein the effective amount of the aqueous microcapsule composition is applied to a surface of human foot skin.

15. An aqueous-based personal care product comprising the aqueous microcapsule composition according to claim 1.

16. The aqueous-based personal care product according to claim 15, wherein the personal care product is selected from the group consisting of shampoos, hair rinses, body washes, soaps, anti-perspirants and deodorants.

17. The aqueous-based personal care product according to claim 16, wherein the personal care product is a roll-on deodorant or antiperspirant product.

18. The aqueous-based personal care product according to claim 16, wherein the personal care product is an emulsion-based deodorant and/or antiperspirant stick.

19. The aqueous-based personal care product according to claim 16, wherein the personal care product is a deodorant and/or antiperspirant aerosol product.
